(19) United States
(12) Patent Application Publication
(10) Pub. No.: US 2008/0266118 A1
(43) Pub. Date: Oct. 30, 2008

(54) Personal emergency condition detection and safety systems and methods

(76) Inventors: Nicholas J. Pierson, Tallmadge, OH (US); Ned A. Seibert, Breckville, OH (US)

Correspondence Address: Edwin W. Oldham, 400 Park Shore Drive, #200, Naples, FL, 34103, US

(21) Appl. No.: 12/075,025

(22) Filed: Mar. 7, 2008

Related U.S. Application Data
(60) Provisional application No. 60/906,043, filed on Mar. 9, 2007.

(51) Int. Cl. G08B 23/00; A61B 5/08

(52) U.S. Cl. 340/573.6; 600/529; 600/508

(57) ABSTRACT

A drowning or asphyxiation prevention system and methods are set forth, for facilitating drowning prevention of swimmers in bodies of water, such as pools, lakes or the like. The drowning prevention safety system comprises a wearable article worn by a swimmer, an alarm indicator for transmitting an alarm condition. The system may further include an alarm receiving system for receiving the alarm signal from the alarm transmitting device. The invention also relates to a heart rate monitoring system comprising a patch type portion to be adhesively applied to the skin of a user to monitor the electrical activity of the heart and generate heart rate information that is communicated to a separate wearable device, such as a wrist worn device.

### CROSS-REFERENCE TO RELATED APPLICATIONS/INCORPORATION BY REFERENCE

This U.S. patent application claims priority to and the benefit of U.S. Provisional Patent Application Ser. No. 60/906,043 filed on Mar. 9, 2007 which is incorporated herein by reference in its entirety.

### FIELD OF THE INVENTION

This invention relates generally to personal safety systems and methods, and more particularly, to a systems and methods for monitoring a person and identifying a person experiencing an emergency physical condition. In one more particular aspect, the invention is directed to a personal emergency system and method for detecting a swimmer that may be in initial stages of drowning, or a person having an asphyxic event, and for commencing emergency response and rescue activities. The personal safety system and methods provide detection of a possible drowning person or person experiencing asphyxia, using heart rate or pulse rate monitoring, or the monitoring of other physiological systems that indicate a person in distress or drowning, or in other cases of asphyxia. The system may include a monitoring system and alarm indicator or transmitter system worn by a person. A variety of other functions may be provided, such as location monitoring, sound monitoring, data recording, and other desired functions. The system and methods may further include an alarm receiver system, located relative to the person wearing the monitoring system, such as relative to a body of water, wherein an alarm indication is detected and communicated to emergency response or safety personnel, or others.

### BACKGROUND OF THE INVENTION

The dangers of accidental drowning are recognized, with parents keeping their children away from the water unless under constant supervision, and attempting to teach their children to swim as early as possible. Yet, drowning is the second leading cause of accidental death in children, and drowning related injuries are the fifth most likely cause of accidental death in the United States presently. Drowning accidents can occur in all age groups, but particularly is of concern with children between the ages of 1 and 4 years old and in teenage children, and many times can be caused by non-supervision, horseplay, and daredevil stunts. Other factors, such as alcohol or other impairment can also lead to drowning accidents. Near-drowning accidents are described as survival after suffocation caused by submersion in a liquid. Drowning accidents are described as events in which a victim dies within 24 hours after having been submerged in a liquid. Generally, when a person becomes submerged, they hold their breath until they cannot do so any longer, with the time for this to occur being dependent somewhat on that person. If the person is still conscious, they may try to gasp for air, aspirating water into the lungs. For many facilities, such as lakes, pools, beaches or the like, lifeguards are hired to attempt to prevent injuries or drownings, requiring significant expense and expertise. Even with lifeguards on duty, drownings still occur each year.

Although there have been attempts to prevent drowning in pools or the like, particularly where there may be no lifeguard on duty, such attempts have not gained acceptance, as they have been expensive and/or ineffective for many applications. For example, anti-drowning systems have included devices carried by a non-swimmer that signal a receiver upon contact with the water. Although assisting where a user is attempting to keep a person out of the water, such as a small child, such devices are limited and don't assist while a person is swimming. Other systems have been developed for pools which use an array of cameras and sophisticated software to attempt to detect an unmoving person under the water, such systems being expensive and prone to difficulties in use.

It would be desirable to more effectively monitor people for water safety to facilitate preventing drowning or other cases of asphyxiation accidents, with a simple but effective system to accurately and quickly identify a possible drowning person or person suffering an asphyxic event, and provide notification of an emergency condition. The identification of an asphyxic event, such as a drowning person, as quickly as possible is important, as death can occur in just a few minutes. A simple, reliable, compact and economical device and methods are needed. It would also be desirable to be able to detect other emergency situations, such as choking, apneaic events or the like.

### SUMMARY OF THE INVENTION

The present invention in one aspect is directed to drowning prevention systems and methods, wherein a system may comprise a device worn by a swimmer that monitors one or more physiological functions of the wearer to identify a potentially drowning person, and provides an indication, such as activating an alarm or a signal for detection of the drowning person. The physiological functions that may be monitored may include pulse or heart rate, blood oxygen levels, peripheral vasoconstriction, body and/or environmental temperature, blood pressure, CO2 shock or other parameters which vary according to the physiology of drowning or asphyxia victims. The system may further comprise one or more detectors for detecting an alarm signal from the device, and facilitating emergency response or rescue activities. The device may further comprise a locating system, such as proximity detection, GPS location system or other system for location surveillance or distance detection of the alarm indication. The device may also include a panic button for the user to trigger the indicator and/or alert a surveillance or alarm detection system, which may also be used to turn off the alarm indication if necessary. The system may also include other features, such as multiple physiological function detectors and/or multiple alarm indicators, such as audible, visual or other indicators. Alarm detection may be provided by various systems, such as one or more sound receivers, visual alarm detectors, or other systems. Emergency conditions may also be detectable from audible sounds of a person that may be in distress or carbon dioxide shock. A visual alarm indicator can be for example a bright light, inflatable balloon type device, colored fluid ejection or the like, may be provided. Other systems to prevent impaired people from entering the water, such as an alcohol monitor or measurement device may be provided. The monitoring system may be small, compact, durable, and easily worn by a person while swimming, or otherwise making it easily usable.

The invention is also directed to methods of detecting an emergency condition and indicating a potential drowning or asphyxia victim, comprising the steps of monitoring one or more physiological functions of a user and determining when one or more functions are outside of predetermined parameters, and indicating this to personnel for initiating emergency response or rescue activities.

These and other advantages of the present invention will be apparent from the detailed description taken with reference to accompanying drawings.

### BRIEF DESCRIPTION OF THE DRAWINGS

FIG. 1 is a perspective view of an example of the system for monitoring physiological systems of a wearer to determine a drowning or other emergency situation and, provide an indication thereof;

FIG. 1A is a perspective view of an example of the system for monitoring physiological systems of a wearer to determine a drowning or other emergency situation and provide an indication thereof;

FIG. 1B is a perspective view of an example of the system for monitoring physiological systems of a wearer to determine a drowning or other emergency situation and provide an indication thereof;

FIG. 2 is a schematic diagram of a system for detecting and issuing an alarm in the event of an emergency condition;

FIG. 3 is a diagram of a method for detecting and indicating a drowning or other emergency situation of a person;

FIG. 4 is a schematic view of a body of water and alarm detection system associated therewith.

FIG. 5 is a view of an example of a personal alarm indicator.

FIG. 6 is a partial view of a visual alarm indicator deployed in association with an example of the drowning prevention system.

FIG. 7A is a side view of the monitor as shown in FIG. 7.

FIGS. 8A-8D are views of monitoring systems according to examples, as worn by a user.

FIG. 9 is partial diagram of another example of a physiological condition monitoring system according to the invention, as worn by a user.

### DETAILED DESCRIPTION

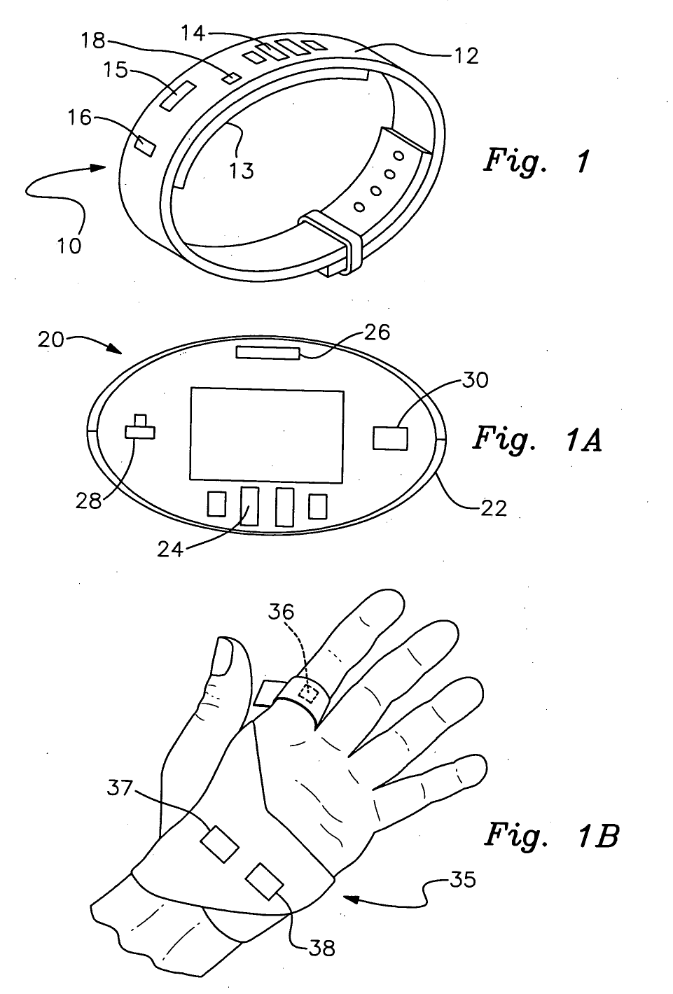

To provide a system and method for detecting and indicating a possible drowning or asphyxia victim, an example of a wearable article or device 10, worn by a swimmer, is shown in FIG. 1. The device 10 may be a wearable device, which is designed to monitor one or more physiological conditions of the wearer. As shown in this example, the device 10 may be a wrist worn device, but other wearable articles are contemplated, such as a patch type device, chest strap type of device, arm, ankle or leg strap type device, ear lobe clip, fingertip clip, ring device, or any other suitable wearable device which can be positioned to monitor the one or more physiological conditions of the wearer. Depending on the physiological function being monitored, the wearable device characteristics may be adapted for effectively monitoring such functions. In this example, the physiological function monitored is the heart rate or pulse rate characteristics of the wearer. The monitoring of the heart rate or pulse rate, provides an indication of a potential drowning victim or person having another life threatening condition. It has been found that in the event a person experiences difficulties while swimming, various indications of an emergency condition may be provided by changes in heart or pulse rate. Although it is common that people experience different heart rates while swimming, due to exertion, excitement, or other factors, more particular changes in heart rate may indicate a more serious situation. According to this example, the device 10 may have a heart or pulse rate monitor and processing functions associated therewith.

As an example, apparatus 10 may be capable of monitoring heart or pulse rate via an integrated heart or pulse rate monitor, alone or in association with other physiological monitoring systems, such as blood oxygen level monitoring and/or other monitors, and a processing system and associated components, such as one or more timers and other components connected to the processing system. The device 10 may further include a strap 12 to allow a snug fit on a user's wrist in order to properly monitor heart or pulse rate or other physiological function. The strap 12 may also be formed of a conductive polymer to enhance the reception of heart rate signals by multiple electrodes associated with the apparatus 10, provided for contacting the user and receiving electrical signals for example. Alternatively, the apparatus 10 may have a conductive layer 13 provided on or selectively attached to the device 10 to provide electrical connection to the user when the device 10 is worn. Depending on the age of the user, the size of the device may be suitably configured. To facilitate detecting electrical signals associated with the heart action of the wearer, highly sensitive electrodes may be used. Alternatively, the detection system may be configured to monitor either the physical movement of a portion of the body, such as at the wrist location, by means of a highly sensitive pressure or touch sensing system, such as a film constructed of self-assembling layers of gold and semiconducting cadmium sulfide nanoparticles separated by insulating sheets of organic compounds, which when a voltage is applied therethrough, converts force applied on to it to a detectable signal. Such materials have been developed and described for example in an article entitled “High-Resolution Thin-Film Device to Sense Texture by Touch”, Science 9 Jun. 2006, Vol. 312. no. 5779, pp. 1501-1504 DOI: 10.1126/science.1126216, which is hereby incorporated herein by reference. Such a sensing system has been shown to have similar sensitivity to that of the human finger, allowing the pulse to be monitored by monitoring the movement of the portion of the body to which the film is applied, similar to measuring pulse with the fingers of your hand. Similar materials may also be suitable for measuring the electrical impulses of the heart to monitor heart rate rather than the movement of the skin due to the pulse. A sensing system using such a material may therefore be sensitive enough to measure the pulse rate at locations such as the wrist, or other locations on the body where the pulse is detectable. Alternatively, other sensing systems, such as described in U.S. Pat. Nos. 6,970,731 and 6,687,523, relating to a fabric-based sensing systems for monitoring vital signs, may be usable in a modified form, for monitoring the heart rate or other physiological functions as desired, which are also incorporated by reference herein.

One or more alarm indicators may be provided with device 10, such as an audible alarm indicator 14 and/or a visual alarm indicator 15, which are coupled to the processing system to receive an alarm signal in the event of emergency condition, and indicate an alarm. The visual indicator 15 may be an extra bright LED or other suitable bright light source, which can be flashed in the event of an emergency alarm. The heart rate monitor may be configured to constantly or intermittently monitor or measure the user's heart rate. Also, dependent on the age or other characteristics of the user, the heart rate monitor may be set accordingly, to more accurately determine a possible emergency condition. A switch 16 may be used for configuring the heart rate monitor to the user, such as based on age or other factors. Alternatively, the heart rate monitor and processing system may be recalibrated automatically as will be described in more detail hereafter, to adjust to the wearer and their heart rate characteristics. The device 10 may also include user activated panic switch 18 that can be selectively activated by the user in the event of an emergency condition and/or to turn off the alarm indication if necessary. The panic switch 18 may be operated in conjunction with the processing system to only allow activation if a potential emergency condition exists as detected by other systems, to avoid undesired alarm indication. Other attributes such as an LCD display, user interface, radio frequency or other wireless alarm communication system, barcode or radio frequency identification, time indication or the like, which are not shown, may be incorporated into device 10.

Alternatively to a wrist worn device 10, as shown in FIG. 1A, the device 20 could be formed as a patch or disk type device, having an attachment mechanism 22 to be selectively attached to the body. The attachment mechanism 22 may be an adhesive layer or a suction type of system to be selectively attached to a portion of the body as an example, or may be attached to a garment worn by the swimmer or other person, such as by a clip or the like. The attachment mechanism 22 may also be a strap to attach to a portion of the body. Any suitable configuration to attach to a user's body and monitor the heart or pulse rate is contemplated. In such an embodiment, the device 20 may also include an alarm indicator 24, and/or a visual alarm indicator 26 may be provided, which are coupled to the processing system to receive an alarm signal in the event of emergency condition, and indicate an alarm. The visual indicator 26 may be an extra bright LED or other suitable bright light source, which can be flashed in the event of an emergency alarm. A switch 28 may be used for configuring the device or heart rate monitor to the user, such as based on age or other factors. The device 20 may also include user activated panic switch 30 that can be selectively activated by the user in the event of an emergency condition and/or to turn off the alarm indication if necessary. Other attributes such as an LCD display 32, a user interface, radio frequency or other wireless alarm communication system, barcode or radio frequency identification, time indication or the like, which are not shown, may be incorporated into device 20.

In either example of the device 10 or 20 shown, other attributes may be incorporated, such as a mechanism to prevent unwanted movement of the electrodes or other monitoring components relative to the wearer. Further water occluding systems may be incorporated to alleviate any effect of the water in which it may be used. As mentioned earlier, depending on the type of physiological function being monitored, the monitoring system may be configured to properly monitor the function. As an example, a blood oxygen monitor may monitor the oxygen level in the wearer's blood, which may be through a finger, earlobe, or other suitable location. Alternatively to electrical impulse monitoring of heart rate, pulse rate may be monitored via movement of the skin at various locations, or detecting the sound of the pulse through the arteries or veins via a microphone system. Other physiological functions may be monitored by suitable systems, such as sound monitoring of response to carbon dioxide shock, peripheral vasorestriction or other physiological responses to asphyxiation, body temperature monitoring, blood pressure monitoring or the like.

Another alternative is shown in FIG. 1B, wherein the device 35 may have a wrist type strap, with an IR sensor to monitor heart rate positioned in association with a finger or wrist of the user for example. In the device shown, a sensor 36 may be positioned on a finger in association with a strap, such as set forth for example in U.S. Published Patent Application No. 2006/0069319, which is hereby incorporated by reference herein. The device 35 may alternatively be attached to another portion of the body where the IR sensor can monitor heart rate from other than the wrist as shown. In this example, the device 35 may further include an additional monitoring system, such as a blood oxygen monitoring system, which may also use the same IR sensor 36. The oximeter may indirectly measure the oxygen saturation of blood at the location of the finger or other suitable location (as opposed to measuring oxygen saturation directly through a blood sample), along with changes in blood volume in the skin. This, in conjunction with a heart rate monitor or other monitoring systems, may provide other data as to the wearer's physical situation to issue an alarm if a potential emergency condition is experienced. Any suitable configuration to attach to a user's body and monitor the heart or pulse rate, the blood oxygen level or other parameters, is contemplated. In such an embodiment, the device 35 may also include an alarm indicator 37, and/or a visual alarm indicator 38 may be provided, which are coupled to the processing system to receive an alarm signal in the event of emergency condition, and indicate an alarm. The audible indicator 37 may be a loud alarm or the like, while the visual indicator 38 may be an extra bright LED or other suitable bright light source, which can be flashed in the event of an emergency alarm. Other aspects of the devices previously described may also be provided if desired, such as a switch for configuring the device or heart rate monitor to the user. The device 35 may also include user activated panic switch 39 that can be selectively activated by the user in the event of an emergency condition and/or to turn off the alarm indication if necessary. Other attributes such as an LCD display, a user interface, radio frequency alarm communication system, barcode or radio frequency identification, time indication or the like, which are not shown, may be incorporated into device 35.

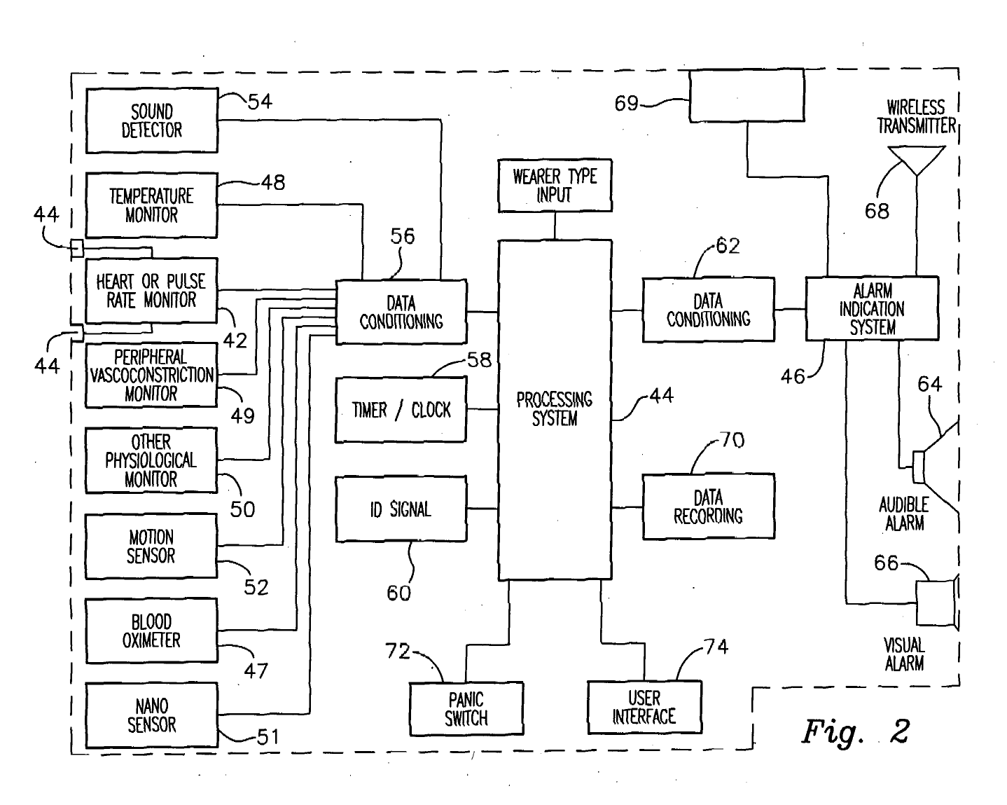

The functions of the examples of the wearable article or device 10, 20 or 35 or others according to the invention for example, will be described in more detail with reference to FIG. 2. The apparatus include a waterproof housing 40 having the one or more physiological function monitors, such as a heart or pulse rate monitor 42 provided therein. In association with heart rate monitor 42, contacts 44, may be provided to receive electrical signals associated with the heart beat of a person for example. A heart rate monitor 42 may be of the EKG type, which picks up an EKG signal from the heart muscle, and calculate the heart rate from the EKG signal. Another example of EKG type system use a chest strap with electrodes properly oriented on the chest strap to pick up the EKG signal directly from the heart muscle. In such an example, the EKG signal may be mixed with a carrier and transmitted to a device, for example, worn on a belt clip, on the wrist or otherwise if desired, or no such transmission is necessary if a system according to the invention is integrated into the chest worn device. Further in such an example, the mixer and transmitter may be located in association with the monitoring device, such as on the chest strap, and transmission is by frequency modulation or any other type of modulation of the carrier by the EKG signal. Any other suitable heart rate or pulse rate monitor 42 is contemplated in the invention, such as a tonometer sensor pulse rate monitor, or any other suitable system. Types of suitable pulse rate monitors for example may also include mechanical systems or the use of visible or infrared radiation which is projected through the skin to detect pulsation of blood flow by radiation reflected from or penetrated through capillaries under the skin. Other heart or pulse rate monitoring techniques may also be suitable. Detection of the sound of the pulse and blood flow through the arteries, veins or capillaries of the body is also contemplated.

The heart or pulse rate monitor 42 provides signals to the processing system 44. The processing system 44 executes analysis of the detected heart or pulse rate, such as by performing various calculations, comparisons and judgments in accordance with a suitable software program, to determine a possible emergency condition. Upon determining a possible emergency condition, the processing system 44 may provide an alert signal to an alert indication system 46. Although heart or pulse rate are used as an example of the physiological processes monitored, other processes or activities may be monitored either in combination or separately. These include for example a blood oximeter 47, temperature monitor 48, peripheral vasoconstriction or larygospasm monitoring system 49, nano touch/pressure sensor 51, or other physiological monitoring system 50, which may include for example a blood pressure monitor, CO2 blood level monitor or other physiological process monitor. As further alternative examples, the system may include a motion monitor 52, which may detect erratic movements indicative of a struggling swimmer for example, or a sound detector 54 to detect sounds associated with a potential drowning victim. It should be clear that either predetermined physiological processes or the predetermined environmental parameters such as those noted, or other suitable parameters related to a potential drowning or asphyxia victim, are contemplated, which may be used independently or in combination. The various monitoring systems may be connected to a suitable data conditioning system 56 if needed, and to the processing system 44.

Other data may be provided to the processing system 44 to support determination of an emergency condition or for other purposes, such as one or more timers or clocks 58, identification data 60, or other supporting components. In association with a heart or pulse rate monitor 42 for example, the timer(s) 58 may be used to monitor the physiological changes in heart or pulse rate, blood oxygen level, temperature, blood flow or content characteristics, etc., over predetermined periods of time to indicate an emergency condition. The identification data 60 may be used to identify the device for transmission in an alarm communication for example. Other systems and components for facilitating monitoring and/or processing are contemplated.

Similarly, the processing system 44 may output an alarm indication signal or other information or parameters for further data conditioning at 62, if necessary. The alert indication system 46 may be of any suitable type, including an audible alarm 64 or a visual alarm indicator 66 for example. The audible alarm 64 may be a high decibel alarm signal (over 80 dB) and/or a mechanical actuator to issue an audible alarm signal from a mechanical knocker or the like for example, while visual indicator may be a high intensity LED for example. Alternatively or in combination, a wireless transmitter 68 may be provided to send an alarm signal to one or more personal alarm indicators as will be described hereafter, and/or one or more receivers/transceivers located in or adjacent the body of water or nearby, for example in the house with a pool or to a remote location. The wireless transmission of an alarm signal may be broadcast using any suitable techniques, such as by radio frequency. An ultrasonic transmitter 69 may also be provided if desired, to communicate an alarm signal to one or more suitable receivers or the like. Any suitable alarm indication to alert safety personnel or others to a possible emergency situation with a swimmer or other person is contemplated. The alarm indication could also be communicated via wireless or wired network, to emergency safety personnel such as EMS, police, fire or the like. The system may further provide a panic switch 72 which can be actuated by the user in case of emergency, or can be used to turn off an alarm signal if needed. In an example, the panic switch 72 may only be operable if there is a coinciding possible emergency condition indicated. This would prevent accidental or unwarranted indication of an alarm signal, thereby preventing “false” alarm indications. A separate turn off or reset may be provided if desired. A user interface 74 may also be provided if desired, for programming or other functions. For example, although a switch 16 or 28 was described in devices 10, 20 or 35 as examples of the system, the parameters for monitoring physiological and/or environmental conditions or parameters can be selectively set via the user interface 74 if desired. The device according to the invention may also use an RFID tag that sends a signal upon being interrogated, such as for identification and security purposes for example.

As an example, the processing system 44 and/or data conditioning system 56 may conduct signal processing functions to identify a possible emergency condition, such as via the heart or pulse rate, blood oxygen level or the like, and may perform processing functions such as amplification, noise reduction, filtering or other processes or techniques. In an example, the system may continuously or intermittently monitor heart or pulse rate, blood oxygen or the like, to detect, amplify, and produce a signal output that is in direct accordance with the subject's heart or pulse rate, blood oxygen level or other parameter. The processing system 44 may also then function to analyze the detected rate or other information, and use this and/or other physiological or environmental information to determine a possible emergency condition of the wearer. This analysis by the processing system 44 may comprise for example, the indication of a possible emergency condition and alarm condition based on various heart or pulse rate variations that occur in possible drowning victims. The physiological effects of drowning have been studied, and continue to be studied, with the invention monitoring the physiology of a potentially drowning swimmer or other instances of asphyxiation to identify potential emergency situations while swimming or otherwise. Data acquired via the system of the invention may provide further information, which in actual use, may facilitate refining the early detection of the physiological processes, environmental or other conditions relating to potential drowning or asphyxiation situations, or other emergency conditions. Data from the processing system 44 may be recorded via a data recording system 70, if desired.

As is known, drowning is death caused by suffocation when a liquid causes interruption of the body's absorption of oxygen and asphyxia. The primary cause of death is hypoxemia and acidosis leading to cardiac arrest. The invention monitors the physiological functions of a swimmer or other person to detect possible near drowning situations, where a swimmer or other person is potentially in danger of drowning or suffocating. As near drowning can involve unconsciousness or water inhalation, the ability for the device 10, 20 or 35 to issue an alarm to nearby personnel, or via a wireless system to other emergency or medical personnel, can facilitate emergency response or rescue in such a situation, by possibly gaining needed attention by other people more quickly.

In water, submerging the face in water generally triggers the mammalian diving reflex, where the body automatically puts itself into an energy saving physiology. The effect of this reflex is heightened in colder water. The reflex generally has certain effects on the physiology, including bradycardia, where the heart rate slows. In humans, a slowed heart rate of up to fifty percent can occur upon occurrence of the mammalian diving reflex. Additionally, such effects as peripheral vasoconstriction, being a restriction of the blood flow to the extremities, which in turn increases the blood and oxygen supply to the vital organs, especially the brain may be caused. Further, depending on the conditions, this reflex results in the movement of blood to the thoracic cavity. The reflex action is automatic and allows both a conscious and an unconscious person to survive longer without oxygen under water than in a comparable situation on dry land. Using the device 10, 20 or 35 according to the invention, the physiology associated with this reflex may be monitored and used to trigger the alarm indication or prevent the indication of a “false” alarm if the reflex is followed by further indications of normal heart function.

While swimming, a conscious person will hold their breath during normal swimming activities, and the invention is designed to monitor and assess whether other than normal swimming activities are encountered, resulting in a potential emergency condition. In a possible emergency condition, if the swimmer or other person is experiencing asphyxia, they will try to access air, often resulting in a panic situation. In this situation, the heart rate can quickly rise in a tachycardia event, or quick increase in heart rate, as a result, as well as other actions such as rapid body movement. An increased heart rate may therefore reflect the physiological as well as the psychological stress level of the wearer, with a rapid increase of the heart rate possibly due to sudden stress, may be due to a physiological condition, such as a cramp, or a psychological stress, such as the fear of drowning. Thus, heart-rate monitoring can help to identify these situations by careful analysis of changes in the heart rate. A rapid heart rate also uses up more oxygen in the blood stream and reduces the time to unconsciousness. Tachycardia is the rapid beating of the heart, which according to the invention, can be the basis of detecting a possible emergency condition, as will be hereinafter described in more detail. The use of oxygen in the blood stream may be detectable by blood oximetry. The rapid movement of the person may also indicate such a condition in conjunction with heart or pulse rate monitoring, blood oximetry or other physiological indicators. Asphyxia is a condition of severely deficient supply of oxygen to the body that arises from being unable to breathe normally. Asphyxia causes generalized hypoxia, which primarily affects the tissues and organs most sensitive to hypoxia first, such as the brain, resulting in cerebral hypoxia. In any case, the absence of effective remedial action will very rapidly lead to unconsciousness, brain damage and death. The time to death is dependent on the particular mechanism of asphyxia. A person can voluntarily hold his or her breath for some time, but the breathing reflex will increase until the victim tries to breathe, even when submerged. The breathing reflex in the human body is related to the carbon dioxide levels. During apnea, the oxygen in the body is used by the cells, and excreted as carbon dioxide, thereby quickly raising the carbon dioxide levels. This increase of carbon dioxide levels leads to the breathing reflex, up to the breath-hold breakpoint, at which point the person can no longer hold their breath. For the normal adult, this typically occurs in a range of about 80 seconds from beginning to hold the breath, but can vary from individual to individual, such as based on the fitness level of the person. This time can also depend on whether the person is already winded from exertion or other factors. Children, with less lung volume, cannot hold their breath nearly as long. If water enters the body's airway at that point, the body's reflex is to close the larynx, and protect the lungs. If the person is still conscious, they may try to cough up the water or swallow it thus inhaling more water involuntarily. Upon water entering the airway, both conscious and unconscious victims experience laryngospasm, where the vocal cords constrict and seals the air tube to the lungs. Although this prevents water from entering the lungs, due to this laryngospasm, water enters the stomach in the initial phase of drowning and very little water enters the lungs. Unfortunately, this can interfere with air entering the lungs, too. In most victims, the laryngospasm relaxes some time after unconsciousness and water can enter the lungs causing a “wet drowning”. However, about 10-15% of victims maintain this seal until cardiac arrest, this is called “dry drowning” as no or little water enters the lungs. If the person is still conscious, hypoxia, or the lack of oxygen to the brain, will quickly result in unconsciousness. This may occur around a blood partial pressure of oxygen of 25-30 mmHg. An unconscious victim rescued with an airway still sealed due to laryngospasm stands a good chance of a full recovery. The conditions will soon also result in cardiac arrest, with the heart stopping. The brain will die after approximately six minutes without oxygen, depending on the conditions such as water temperature. If water is aspirated in freshwater which contains less salt than blood, it will therefore be absorbed into the blood stream by osmosis. In animal experiments, this was shown to change the blood chemistry and led to cardiac arrest in 2 to 3 minutes. Sea water is much saltier than blood. Through osmosis water will leave the blood stream and enter the lungs thickening the blood. In animal experiments the thicker blood requires more work from the heart leading to cardiac arrest in 8 to 10 minutes. However, autopsies on human drowning victims show no indications of these effects and there appears to be little difference between drowning in salt water and fresh water. The monitoring of the physiological functions of a swimmer allows the alarm indication of a possible emergency condition to be made prior to these more serious consequences.

As mentioned previously, in addition to monitoring physiological functions such as heart or pulse rate, and/or blood oxygen or carbon dioxide levels, the device 10, 20 or 35 may utilize the other physiological responses to detect initial and further stages of drowning, such as monitoring and detecting peripheral vasoconstriction via a monitor 49. In response to suffocation or drowning, peripheral vasoconstriction may be monitored by a loss of blood flow to the extremities or other physiological parameters related to this process It is also found that other parameters such as body temperature may indicate a potential emergency condition. For example, the temperature of the body begins to decrease immediately upon holding the breath when swimming. Further temperature drop occurs upon continued oxygen deprivation, and monitoring the temperature over a predetermined time period may therefore be an indicator of asphyxia. The temperature monitor 48 may therefore be used to detect body temperature and/or environmental temperature for an indication of possible asphyxiation or other emergency condition. For example, in some situations, such as in a lake or the like, the temperature of the water decreases significantly upon getting deeper. Any atypical exposure to significantly lower temperatures in the environment may indicate a person has been submerged for too long and may need assistance. Decreased body temperature, even in a colder environment, over a predetermined period of time may also indicate asphyxia or potential drowning. For example, a decrease of several degrees in body temperature over a short period of time may be an indicator of a problem. Other physiological processes or parameters, such as blood oxygen, blood pressure, blood CO2 levels or other parameters, may also be monitored by 50 in a manner to indicate a possible emergency condition. Further, the device 10, 20 or 35 for example, may include a motion sensor 52 to detect uncontrollable movement of arms or legs, which sometimes occurs in a panic situation or emergency condition. Further, a sound detector 54 may be used to detect the sound of a person aspirating water, or when the breath hold point is reached and the person involuntarily takes a breath or upon the occurrence of laryngospasm. Other sound associated with the physiological response of a potential drowning or asphyxia victim may also be detectable. A combination of monitored physiological functions and/or environmental conditions such as temperature, sound, motion sensing or the like can be used. In an example using heart or pulse rate monitoring, the identifying physiological indicia of a potential emergency condition may be based upon various heart beating responses, such as bradycardia and/or tachycardia indications, or other factors, either alone or in combination. In general, a person's heart functions may be characterized by parameters including heart rate, heart rate variability (HRV), heart rate recovery (HRR) and possibly other characteristics. Heart rate variability is a measure of the variations in heart rate. It may be calculated by analyzing a time series of beat-to-beat intervals of heart function. Various measures of heart rate variability have been proposed, which can roughly be subdivided into time domain, frequency domain and non-linear measures. Alterations, such as reductions, in HRV have been reported to be associated with various pathologic conditions that may indicate an emergency condition. Heart rate recovery (HRR) is the heart rate that our body will decrease to after an elevated rate is experienced. For example, if the heart rate rises to above the resting rate, recovery to the resting rate occurs over time in a normal fashion if no exertion or other factor leading to an elevated rate continues. HRR may be measured over a 5 to 15 second sampling interval for example. A drop of 20 beats in a minute is typical for a healthy person for example, but can vary.

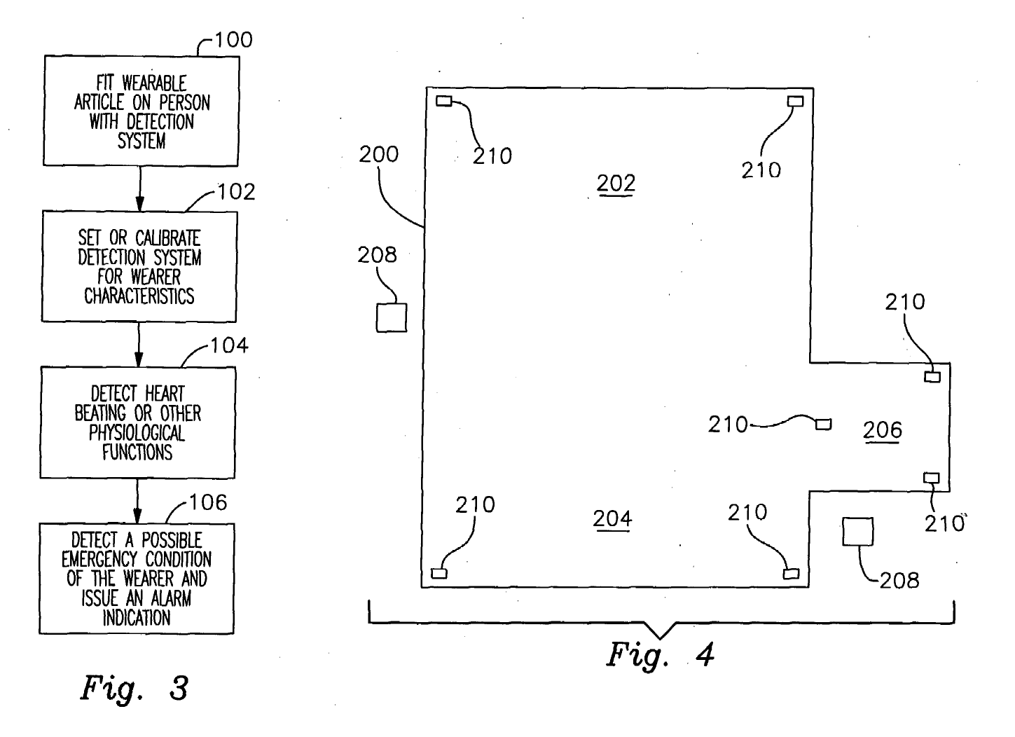

In use of heart rate data over time, the indications of an emergency condition may vary based upon the age, or factors such as fitness, heart disease or other physical problems, or other factors. According to an example of the invention, the switch 16 or 28, or other user interface, associated with device 10, 20 or 35 as previously described, may allow the monitoring functions of the device to be adjusted for the wearer, or the device may automatically be adjustable to the characteristics of the wearer. As seen in FIG. 3, the monitoring device is fitted to the user at 100, and is set or calibrated to the characteristics of the wearer at 102 to facilitate proper detection of a possible emergency situation. The heart beat is monitored for processing at 104, such as by device 10, 20 or 35, and the processing system 44 and associated software uses the heart or pulse rate information to determine a possible emergency condition at 106.

Using bradycardia symptoms for example, the device may indicate a possible alarm condition if the heart rate falls at a predetermined rate over a predetermined time period or based upon a drop of heart rate below a predetermined threshold. To detect a possible emergency condition as soon as possible, a predetermined drop in heart rate over a predetermined amount of time may indicate a person experiencing a hypoxic event. In an example, a condition which may be termed profound or terminal bradycardia may indicate an emergency condition. For example, profound or terminal bradycardia may be considered a predetermined drop of heart rate in a given time period. For example, terminal bradycardia may be indicated by a drop of heart rate which is greater than the time period elapsed, for example of drop of more than one beat per second. In an example, the device may monitor heart rate over time, and indicate a possible emergency condition upon detecting a drop in heart rate of 10 or more beats per a ten second period. As an alternative, to avoid possible “false” alarms, the device could indicate a possible emergency condition upon detecting a drop of 15 or more beats per a ten second period. Other factors, such as whether the person is already oxygen depleted, such as from exercise or the like, may result in terminal bradycardia indications more quickly.

Also, in response to a hypoxic insult, a person's heart rate may drop to less than their minimum normal heart rate which lasts for a predetermined period of time. For example, for a typical adult, a rate of 60 bpm or less over a period of time, such as 10-20 seconds, may indicate a possible emergency condition. A lower heart rate threshold may be used to indicate an emergency condition, such as below 40 bpm for an adult. For children, who have a higher normal minimum heart rate, a drop below a threshold minimum rate would be indicative of an emergency condition. For example for very young children, a heart rate of under about 90 beats per minute (bpm) over a period of time, may be considered to be in bradycardia, and an alarm condition may be indicated. For older children and teens, a heart rate under about 60 bpm may be considered to be in bradycardia, and an alarm condition may be indicated, such as below 70 bpm for younger children over a period of time, or a threshold of 60 bpm. Other threshold values based on the characteristics of the wearer are contemplated. These are only examples of possible detection of bradycardia symptoms, such as in relation to predetermined reductions in rate over time, heart rate thresholds, or other approaches for detecting an indication of abnormal bradycardia, and any suitable approaches are contemplated. A depressed heartbeat can prevent the brain and other vital organs from getting sufficient oxygen, causing permanent damage, even death, and the invention is directed to detecting such a condition and providing emergency assistance before significant damage or death occurs.

Detection of an emergency condition may also be based upon tachycardia symptoms. For example, an emergency condition may be indicated by detection of profound or terminal tachycardia, which may be considered to be a rise of 50-250 bpm in 10-20 seconds, based on the characteristics of a person. Other parameters, such as a continued rate above a threshold rate, with little, abnormal, or no heart rate recovery may be used as an indicator. For tachycardia, as an example, for young children, a heart rate over about 220 bpm may be considered to be in dangerous tachycardia, and an alarm condition may be indicated. For older children and teens, a heart rate over about 200 bpm may be considered to be in dangerous tachycardia, and an alarm condition may be indicated. For adults, a heart rate over about 180 to 200 bpm may be considered to be in dangerous tachycardia, and an alarm condition may be indicated. Alternatively, the device may be calibrated according the persons resting heart rate, which may be initially calculated upon putting the monitoring system on, with a threshold for tachycardia based on the calculated value. Alternatively, the indication of tachycardia may be based on an increase in heart rate over time, such as within a predetermined amount of time. As an example, the indication of terminal tachycardia may be based on a predetermined increase in heart rate over a predetermined amount of time, such as an increase of 30-140 bpm over a period of time, such as between 10 to 60 seconds. These are only examples of possible detection of tachycardia symptoms which indicate a potential emergency condition, such as in relation to heart rate thresholds, predetermined increases in rate over time or any suitable approaches are contemplated. Another possible alternative may be to relate the indication of tachycardia to maximum heart rate for indicating an emergency condition. For people, the maximum heart rate may be calculated and estimated for an individual, based on age. For adults for example, the maximum heart rate is estimated to equal 220 bpm minus the person's age.

The system may include multiple timers that work in conjunction with the processing system, to measure heart rate variability, heart rate recovery and/or cardiac rhythm. The timers may operate to produce an alarm in the event that measured parameters as set are out of the normal range for human physiology. For example, the recovery heart rate may be measured based on normal increases in rate due to entering water or exercise, and may be measured over a 5 or more second sampling interval for example. From an elevated rate from the resting heart rate, a drop of 20 beats in a minute is typical for a healthy person, while a drop of less than 5-10 beats per minute may indicate a problem. Heart rate variability (HRV) is the variation of beat-to-beat intervals. A healthy heart has a large HRV, while decreased or absent variability may indicate cardiac disease. HRV also decreases with exercise-induced tachycardia. HRV can be used as a physiological marker to classify a potentially emergency condition. In general, the heart rate and rhythm may be used, alone or in conjunction with further parameters, to determine the emergency condition. The rhythm of the heart is normally generated and regulated by cells within the heart, with normal rhythm generally very regular, with minimal cyclical fluctuation. Furthermore, atrial contraction is always followed by ventricular contraction in the normal heart. When this rhythm becomes irregular, too fast (tachycardia) or too slow (bradycardia), or the frequency of the atrial and ventricular beats are different, this is called an arrhythmia. The term “dysrhythmia” is sometimes used and has a similar meaning. Abnormalities in these conditions may be used to detect an emergency condition.

As an example using multiple timers, the system may be configured to monitor the physiological function and determine within various time ranges, whether any significant abnormalities are present in the physiological functions. In the case of heart rate, the timers may be used to monitor rate changes in the individual, such as over predetermined time ranges. If the heart rate drop is too fast, or rise is too quick, a first timer may be set to indicate a possible bradycardia or tachycardia condition. Thereafter, second or additional timers may be used to continue to monitor the heart rate and determine the variability over additional periods, with an alarm condition being indicated if the variability or parameter is still out of an acceptable range. In an example, the second timer could monitor heart rate starting at a point during the first timers period, to effectively allow the rate measurements to be verified in determining an emergency condition.

For terminal bradycardia detection for example, a predetermined heart rate drop per predetermined period such as 5-15 seconds, may initiate the first timer. For terminal tachycardia, a predetermined rise in heart rate per predetermined period such as 5-15 seconds, may initiate the first timer. The first timer may be configured to operate while monitoring continues over a next predetermined time period, such as 5-20 seconds. A second timer may be initiated at midway through the cycle of the first timer, such as for a similar or different time period, with monitoring continuing. If the measured functions, such as heart rate parameters, are found to be within acceptable ranges during the period of the second timer, the first timer will be reset, and no alarm condition will be triggered. On the other hand, with heart rate measurements, if continued bradycardia or tachycardia conditions are measured during the second timer, the alarm can be triggered. As should be recognized, additional timers may be used to continue to monitor possibly abnormal conditions over additional time periods. Further, if the timers are used and triggered more than once within a given period, this may indicate an alarm condition.

Other parameters such as stressful apnea (voluntary or involuntary apnea), obstructed apnea, heart attacks, seizure or other abnormal conditions may be detected and used to issue the alarm.

The indication of an emergency condition based on bradycardia and/or tachycardia indications over time or thresholds as described, or other parameters of heart function, may depend on the age, physical fitness or other factors of the person for example. The device according to the invention may therefore be set or calibrated for a particular user. For example, the device may be calibrated according the persons resting heart rate, which may be initially set or calculated upon putting the monitoring system on. As an example, when resting, the average adult human heart beats at about 70 bpm (males) and 75 bpm (females); however, this rate varies among people and can be significantly lower in athletes (ie. 40-60 bpm). The infant/neonatal rate of heartbeat is around 130-150 bpm, the toddler's about 100-130 bpm, the older child's about 90-110 bpm, and the adolescent's about 80-100 bpm. The parameters relating to bradycardia and/or tachycardia indications may then be based on the calculated or set resting value. For example, the thresholds of bradycardia and/or tachycardia may be set at a rate about 10-40 bpm lower than or in a range about 100-140 bpm higher than the resting heart rate, such as 120 bpm higher than the resting heart rate. As the resting heart rate can vary between people, for example based on age, fitness or the like, independently determining a persons resting heart rate may be performed to be more accurate. In the above examples, the resting heart rate may be determined initially if the person putting the wearable article has been at rest, but if not, may be determined using a recalibration process wherein the heart or pulse rate is monitored and based upon the range of lowest rates monitored without an emergency condition detected, and this can be used to recalculate the resting heart rate over time.

In these examples, various heart rate physiologies can be used to detect a possible emergency condition, and these can be used in combination to detect different possible indicators. From the foregoing, both bradycardia and tachycardia type symptoms may be an indicator for a drowning or asphyxia emergency condition, and both lower and upper thresholds, changes over predetermined time periods and/or other alarm indicating conditions may be used if desired. For setting the heart rate characteristics and thresholds or other physiological conditions that are monitored, the device may also have a user interface for setting heart rate values or other parameters. Beyond upper and lower heart rates, and a predetermined reduction or increase of the heart rate over a predetermined amount of time, other parameters such as a heart rate indicating a panic situation, a heart rate indicating an unconscious person, or any other heart functions suitable for indicating a possible emergency condition of a swimmer or other person experiencing asphyxia or other distress may be used. Use of this or other physiological changes occurring over time may facilitate providing an early indication of an emergency condition. It has also been found that in episodes of asphyxia by breath holding, leading to terminal bradycardia, there may initially be a significant increase in heart rate, followed by a significant decrease in heart rate, followed by another increase, and a further decrease in rate before beginning to decrease more rapidly in terminal bradycardia. Other physiological conditions may include for example, the heart skipping one or more beats, palpitations, beating out of rhythm, rapid heart action or the like. These or other physiological occurrences as described can be detected and used, either alone or in combination, to detect an emergency condition and issue an alarm indication.

In the case of blood oxygen levels, the monitoring may be conducted similarly, and drops in the level used to detect and/or verify an emergency condition. Timers may again be used to monitor and detect an abnormal condition in a similar manner as previously described, but in relation to the different physiological parameter. Generally, the blood oxygen level should be in the range of 90-100%, with a possible problem indicated with drops below 90% for example.

The processing system 44 as previously described may use sampling/processing cycles to continuously monitor heart rate characteristics, and determine if the measured heart rate value falls outside of either of the upper and lower threshold values indicating bradycardia or tachycardia, or a predetermined variation over time or other heart beat characteristics as described, and issue an alarm indication. The alarm indication can be by any suitable method, such as an audible alarm of significant magnitude, for example 86 dB or above. Alternatively, the indication may be an ultrasonic signal having a predetermined frequency or range of frequencies, or may be visible, such as by use of bright, flashing LED's or the like. Further, an alarm indication may be sent wirelessly, such as by an RF transmitter or cell transmission, which would send an encoded signal to a receiver alert station, including possibly emergency personnel, the police or fire rescue or the like. Upon providing an alarm indication, the system may continuously issue an alarm or repeat the indication repeatedly over short intervals until the unit is manually reset.

In an example of use in a pool or the like, the system of the invention may be worn by swimmers, and any alarm indication then being communicated to lifeguards or others, either at the pool or the like, or at a remote location. As seen in FIG. 4, a pool 200 may be outfitted with an alarm sensing system to facilitate receipt of an emergency condition alarm indication. The pool 200 may have a shallow portion 202, a medium depth portion 204, and a deep end or diving well 206 as merely an example. Lifeguards are typically positioned at several stations around the pool 200, such as at chairs 208. For receipt of an alarm indication, arranged in each of these areas may be a plurality of alarm sensors 210, such as hydrophones, cameras, microphones, signal receivers (such as for RF alarm signals or the like), light detection systems and/or ultrasonic receivers, or other suitable alarm detection systems, which will quickly detect an alarm under or above the water surface. If an audible alarm is used, the volume of the alarm may also be quite high to result in easier identification, particularly where a person extends the device above the water surface. It is also contemplated that the array of alarm sensors 210 could be coupled to a processing system, to identify the location in the body of water from which the alarm came from, such as by triangulation, attenuation or other suitable techniques. The array of alarm sensors 210 may be positioned to adequately cover the body of water being monitored, such as by positioning for detection at both shallow and deeper water locations for example, or at spaced intervals around the perimeter or within the perimeter of the body of water. As described, the alarm detection system may comprise a hydrophone or audible sound sensing system and/or visible alarm sensing system or other alarm sensing systems. In this example, the sound sensing system may be configured to receive sounds generated in the water, and filter the sounds to detect a predetermined frequency audible alarm, for example. The visible alarm sensing system may be a camera having tracing capabilities to detect a visible alarm comprised of a predetermined frequency light for example. The camera sensing system as an example, may be configured to constantly change its view to detect an LED or other visual alarm on the wearable device under the water surface and communicate an alarm condition to the lifeguards or others. The communication could be wirelessly as previously-described, or by other suitable means. Using one or more alarm sensing systems, it is also possible to perform location determination using triangulation or other suitable techniques.

As an example, the receivers or sensors 210 may be sound sensors for detecting an audible or inaudible alarm, with the sensor(s) tuned to the frequency of the audible or inaudible alarm issued for example.

As an example of an inaudible alarm, a wireless signal, such as an RF signal may be produced, or an ultrasonic signal may be produced, with RF or ultrasonic receivers positioned around or in the body of water for detection. The RF or ultrasonic signals may also be transmitted at predetermined frequency or frequency range to be picked up by a similarly tuned receiver, to exclude other possible sources of signals. Another example uses a wireless transmitter using radio frequency or other suitable transmission medium. An RF transmitter may provide an array of RF receivers with an alarm signal for detection. The RF receiver system may include RF detection, amplification, heterodyne, and signal processing hardware required to decode the intelligence carried within the RF carrier wave and produce a indication of the alarm condition to local or remote personnel in any suitable manner, such as by means of a wearable personal alarm indicator described hereafter, or other suitable means such as an audible or visual alarm at the location of a lifeguard, or transmission of an emergency call to the EMS or paramedics for example.

In an example, one or more receivers 210 or a separate alarm location determining system could be configured to receive an alarm, and/or could include a system to detect an alarm and also the location of the person in distress. A sonar type system may be provided in association with receivers 210 that would enable detection of the precise location of the person in distress, which could then be communicated to desired personnel, such as lifeguards, parents or the like. As an example, some sonar type systems may use an acoustic signal/wave sent out and reflected by air pockets similar to fish finders that detect the bladder of fish having air therein. In the present invention, upon issuance of an alarm indication, the monitoring device according to the invention could include an inflatable bladder that would be detectable in a similar fashion. The reflected acoustic signal/wave would be reflected by the bladder and converted to an electrical signal for display as to location. Any other suitable arrangement to allow detection of the monitoring device from which an alarm is being issued in a similar manner is contemplated by the invention. In a sonar type system, sound will be generated by the alarm receiver upon receipt of an alarm indication. The sound signal/wave travels through fresh water at a speed approximately 4920 feet per second, and the amount of time for a burst of energy to travel to the person in distress and be reflected is measured. This time variation may then be displayed on a suitable readout device. As an example, the alarm receiving system may have an electronic power pack which generates very short bursts of electrical energy which are sent to a transducer, which operates as a “loudspeaker” to convert those short bursts, or pulses, of electrical energy into very short bursts of high frequency sound energy. After sending out a single burst of this high frequency sound, the transducer is switched over so that it now acts as a “microphone” to pick up the sounds of the returning echoes created when that pulse of sound hits the monitoring system. The returning echoes of this short pulse of high frequency sound are received back by the transducer (operating as a microphone) which converts sound energy into electrical energy. These tiny bursts of electrical energy, now much weaker than the original signal, may then be amplified to provide an indication of the location of the reflected signal. A bobber type sonar system could be used that transmits a RF signal from a remote location to a suitable receiver, such as a display or handheld device that shows location of received signal.

Alternatively, the alarm receivers 210 may include RF receivers, either worn by a lifeguard or the like or set up around or in the body of water. An RF signal produced by the monitoring system indicating an alarm condition may be of suitable strength such that only local receivers pick up the signal, thereby providing an indication of location of the person in distress. As an example, an RF alarm signal could be produced for reception by the nearest lifeguard(s), while other guards that may be monitoring the body of water would not be alerted at least initially, thereby allowing the location from which the alarm is generated to be generally determined. In environments such as wave pools, lazy river type attractions or the like, providing only a local alarm signal may allow immediate response from a lifeguard in that local vicinity. Another alternative could be the use cameras configured to detect a visual alarm indications from the wearable monitoring devices, with information provided from such cameras used to locate the person in distress and notify the nearest emergency response person, as well other personnel as to the location of the person in distress. Camera systems could be configured to scan the body of water, and automatically detect a light source indicating an alarm condition, and the location of the person in distress, and communicate this information to a lifeguard or the like. As another alternative, the attenuation of sound relating to an audible alarm may provide location information for communication to desired personnel. If multiple receivers are used, the nearest receiver 210 could also provide location information, based on being the first receiver to detect the alarm or triangulation techniques. For a sound alarm for example, the location of the issued alarm could be provided for determining the position of the person in distress, such as by determining the attenuation of the sound signal in the water. Depending on the strength of the received alarm signal from its original strength (which is known), the signal may indicate the relative position of the location of the issued alarm to the sensor 210. Emergency response personnel such as lifeguards on a display or other suitable method. The communication of location information may be made by any suitable systems, such as a display showing the location of the alarm signal and thus the person in distress, a wireless transmission, visible, audible or other suitable indicator associated with the nearest receiver 210 or other suitable systems. Such a display or the like may be associated with a personal alarm indicator described hereafter, worn by lifeguards or other personnel, or a display positioned at the location of a lifeguard or other personnel for example.

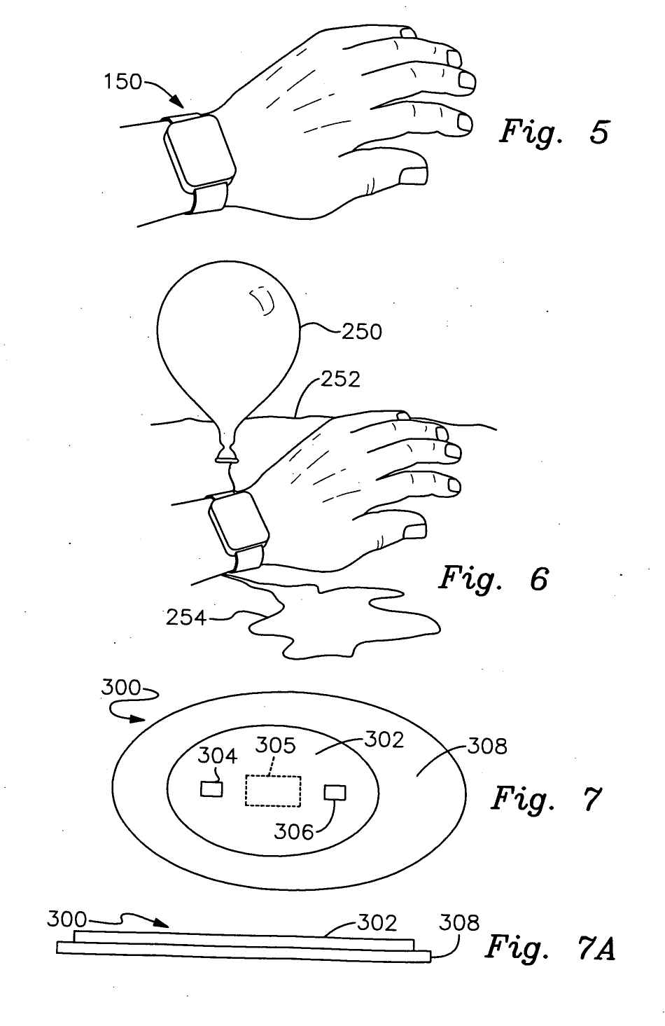

It may be desirable to provide a discrete indication of an alarm to desired personnel. As an example shown in FIG. 5, the alarm may be communicated to a personal alarm indicator 150, such as a wearable device worn by lifeguards, parents or the like, to immediately provide emergency response and assistance in the case of an alarm. To facilitate use in many environments for example, it may be desirable to issue an alarm in a discrete fashion, to avoid possible panic in others. The alarm may be issued by the device in a fashion that is discrete, such as by one or more signals, such as an audible signal that is outside of the normal human hearing range, a visible signal outside of the normal visible light range viewable by humans, a RF signal or other suitable signal source which is not readily detectable by the human senses, or other suitable means. The alarm signal may be issued and detected via a suitable detection system, and indicated to desired personnel via any suitable system. For example, this may be accomplished by providing a wireless communication alarm to a head lifeguard or multiple lifeguards, parents or others via a personal alarm indicator, such as a wearable article or the like. As described, the system of the invention may detect the location of the indicated alarm and person in distress, and this information may be also be transmitted to desired personnel if desired. Depending on the characteristics of the aquatic facility, it may be desirable to communicate the alarm and location of a person in distress to a head lifeguard, who can then initiate emergency response via the nearest lifeguard on duty, via walkie talkie or the like. Such an approach may be suitable for large aquatic facilities such as wave pools or the like. In other environments, such as smaller pools, it may be desirable to indicate the alarm to any lifeguard on duty to allow immediate response. As seen in this Fig., the alarm indicator device 150 could be configured as a watch type unit that includes a vibratory annunciator which alerts the individual of an alarm signal from monitoring devices according to the invention. In an example, the lifeguards around a pool or the like, may be outfitted with a wearable personal alarm indicator, such as a wrist worn device, finger ring or other suitable personal alarm indicator, that has the ability to receive an alarm indication, and notify the wearer of the personal alarm indicator. Any desired personnel, such as lifeguards, parents or others may be provided with such personal alarm indicators. The indicator devices may be configured for example, to be able to send and receive RF signals. Receipt of an alarm from a person in distress (communicated discretely), or from one or more of the alarm receivers, such as receivers 210 that may be used, would provide emergency response as needed. Alarm receivers, such as receivers 210, could be configured receive an alarm indication from a person in distress and in turn to issue an alarm indication to the various personal alarm indicators in the vicinity upon detection of an alarm condition. As in prior examples, the receivers may be configured to be tuned to a specific alarm indication, such as a particular frequency audio or visual alarm signal, RF signal or the like. A plurality of receivers 210 such as previously described, may be used to assist in alerting the nearest lifeguard of other emergency response person, by location detection of the person in distress. The ability to indicate the alarm condition in a discrete manner allows immediate emergency response, but without causing panic in other patrons in an aquatic facility for example. If desired, the alarm indicator device may be tuned to a particular monitoring device to only receive an alarm signal generated by that particular monitoring device, such as for use by a parent or the like.

In accordance with an example of the present invention, a home or private environment drowning prevention safety system is provided. Such a system may be useful for situations where lifeguards are not on duty. The body of water drowning prevention safety system comprises an article worn by a swimmer, an alarm indicator for transmitting an alarm condition, and an alarm receiving system for receiving the alarm signal from the alarm transmitting device. The system may further include a communication device to notify emergency personnel and initiate obtaining medical assistance for the person from which the alarm was issued. The communication system may provide two way communication to allow a central command type facility to communicate with the site of the pool or the like. The system may operate somewhat like a home security system, where issuance or an alarm from a monitoring device worn by a swimmer will be communicated to a central command and initiate emergency response if necessary. The system may also be configured to allow diagnostic testing of the monitoring systems, alarm receiving system or other components of the system to ensure proper operation. Upon issuance of an alarm, the central command will attempt to communicate with persons at the site of the alarm to verify an emergency condition. In this way, if no emergency condition is found, no emergency response will be initiated. Alternatively, if an emergency condition is verified or no response is provided, the central command can immediately send emergency response personnel. The two way communication could also include video monitoring to allow central command to visually monitor the situation at the pool or the like if desired. There may also be provided an alarm signal in the users home if the pool or the like is outside the home, to alert people inside the house of an emergency condition, or a personal alarm indicator may be worn by people in the house and a wireless alarm signal (ie, RF transmission, cell transmission, etc.), could be issued and detected as described previously. The central communication or a computer system could be provided to record information relating to the emergency condition and activation of an alarm or other information. As an example, the system may be configured to communicate with appropriate emergency response personnel, doctors, nurses or the like, such as by cell phone communication, for two way, real time, and communications during an emergency situation. Other communications could also be automatically initiated, such as to facility or management personnel or others, in the event of an emergency situation.

Turning to FIG. 6, there is shown another feature that may be associated with the device 10, 20 or 35 for example. Although the provision of an audible alarm or a visual alarm such as a flashing light has been set forth, it may also be desirable to provide a further alarm indicator for identification of the location of a potential drowning victim. As many people in distress in water will be submerged, a flashing light or the like may not be detectable above the water surface. As shown in FIG. 6, as an example of a further feature, the device 10, 20 or 35 may be provided with a balloon alarm indicator, which is automatically deployed upon predetermined indications of an emergency or alarm condition being met. In this example, a balloon 250 is deployed such as by a carbon dioxide charge or the like, which will quickly inflate the balloon 250 in the event of an emergency condition. If the swimmer or other person is submerged, the inflated balloon 250 will cause the arm of the wearer to be elevated until the balloon is exposed above the water surface 252. The balloon 250 may be brightly colored or the like to allow quick detection and such an alarm indicating feature may be used separately or in conjunction with the other alarm indications as discussed. Alternatively or in addition, a colored fluid 254 may be ejected from the monitoring device upon an alarm indication to provide another indication of the location of the person in distress. The ability to quickly locate a drowning person, by any of the alarm indicators described or other suitable indicators will allow emergency response personnel to react quickly in the event of an emergency condition. If desired, the wearable article 10, 20 or 35 or the like, may also be configured for non-swimmers, to include a submersion detection system for issuing an alarm condition if a non-swimmer goes into the water or becomes submerged. In use of this approach, there may be no need to monitor physiological processes of the non-swimmer. Submersion detection can be performed in any suitable manner, such as by an electrical contact switch that is closed by water, or from a pressure sensor or any other method. The monitoring device may also be usable to monitor infants in prevention of sudden infant death syndrome (SIDS), for choking or asphyxia conditions, home health care, assisted living-nursing homes, public transportation, other health related applications, security companies, sleep apnea, daycare operations, accidental asphyxia, or other applications. Sudden death in infants may result from cardiac arrhythmias, sudden respiratory failure or accidental asphyxiation. The invention may provide an early indication of such problems to allow resuscitation. Obstructive sleep apnea and/or apnea of prematurity may be detected before or upon respiratory failure leading to SIDS, and the present invention may be used to detect such occurrences and alert the parent for emergency response.

Further, the systems according to the invention may be used to facilitate set up and maintenance of the system in association with an aquatic facility or the like. As an example, the device could be set to activate the alarm continuously, and randomly dropped into the water to test receipt of the alarm, location sensing systems and the like.

For use in commercial aquatic facilities, the wearable monitoring device can be provided with a system to be detected by a matched ‘reader’ whenever they are brought within range. This feature can prevent unauthorized removal of the wearable device from the site by warning both the wearer and management. For example, the device may include a RFID tag, to produce a warning in the event that a patron leaves a predetermined area. The effective range of the device may be adjusted as needed for a facility. Alternatively, the facility operator may impose a financial deposit, taken at the point of swim purchase, and refunded when the swimmer leaves the premises. The wearable device can act as a ticket on site and the refund is made at the time of departure. This can be facilitated by allowing users to deposit the devices in a repository unit upon departure. The repository could be configured to detect the device and provide a refund of the deposit.

Turning to FIGS. 7 and 7A, an example of a heart rate monitor which may be used in association with detecting an emergency condition or in other applications is shown. For use in detecting an emergency condition in many environments as described above, it is desired to monitor heart rate while a person is active. Devices have been developed for monitoring heart rate for other purposes, such as during exercise, to enable the user more effectively exercise by maintaining the heart rate in an aerobic exercise level, such as produced by Polar, Garmin, and other companies. There are known monitoring systems that utilize a monitoring device worn adjacent the chest for example, that is positioned by means of a chest strap for example. Such devices monitor the heart electrical activity to measure heart rate, and then transmit the information to a wrist worn device via wireless communication, such as RF signals or the like. Although providing a suitable system for monitoring heart rate, such systems have drawbacks, including the positioning of the device adjacent the chest by a chest strap, which is uncomfortable, unattractive and susceptible to movement, thereby compromising the integrity of the heart rate measurement. Particularly, for use in the water environment, such as in association with drowning detection as set forth in above examples, such systems are deficient in that they may not be waterproof, and again are very unattractive for a person in a bathing suit to wear. Further, the use of a strap may not provide stationary positioning of the device on the wearer, particularly if the person is active in the water, including such activities as diving, active swimming or the like. This example of the invention therefore overcomes these limitations by providing a monitoring system in the form of an adhesive patch, that is more attractive to wear, whether during swimming or any other activities, and positively positions the monitoring system on the chest of the wearer to ensure accurate and proper heart rate monitoring. In this example, the patch system 300 includes a first portion 302 having a pair of electrodes 304 and 306, adapted to be placed into engagement with the skin of the user, such as at an area adjacent the chest or other suitable location. To facilitate electrical contact with the skin, there may be provided reservoirs in association with the electrodes into which an electrically conductive electrolyte gel can be placed before application to the wearer's skin. Interior to the portion 302, the electrodes are connected to a monitoring system and circuit 305 to detect and communicate heart rate information to a wrist worn device for example, similar to the known systems. The portion 302 becomes a reusable portion, that is selectively mated with an adhesive portion 308 having an adhesive area at least around the exterior of portion 302, to allow the combined system to be selectively adhered to the skin of a wearer. The adhesive portion 308 may be configured in any desired shape, such as a heart shape, or a shape appealing to the wearer. As the system may be configured for use with children, the shape of the portion 308 may be appealing to children, for example. The outer surface of the portion 308 may also include graphic or other decoration to make it appealing also, such as emulating a character (Mickey Mouse, etc.) or the like which would be appealing to children. The combined system provides a very low profile, small and conveniently worn system, which positions the electrodes 304 and 306 positively against the wearer's skin, without the possibility of movement or disengagement. The system is waterproof against the skin, and can be formed to be attractive for use in water environments, or for any other environment where use of a chest strap type of system is inconvenient or susceptible to the problems noted above. For example, persons such as firefighters, police, infants or a wide variety of other types of users could benefit from a patch type of monitoring system as contemplated in this example. In use the adhesive portion 308 may be provided in association with reusable portion 302, for use during a period of time, and then the assembly may be removed and the portion 302 subsequently reused in association with the same adhesive portion 308, or a new adhesive portion 308 as may be desired. For commercial applications, the ability to use a new adhesive portion with a new wearer would be beneficial. As an alternative to electrodes 304 and 306, a highly sensitive pressure or touch sensing system as previously described, may be used.

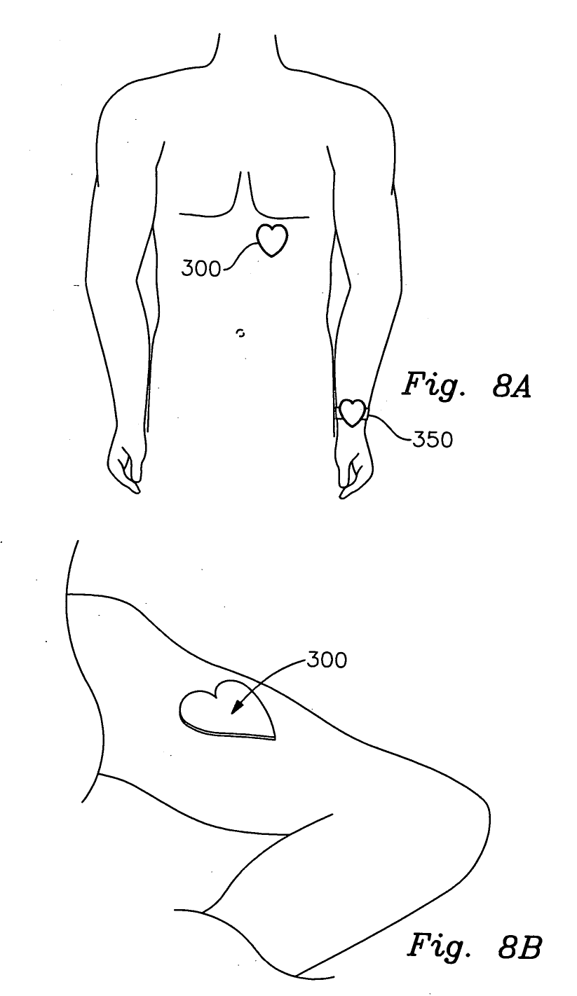

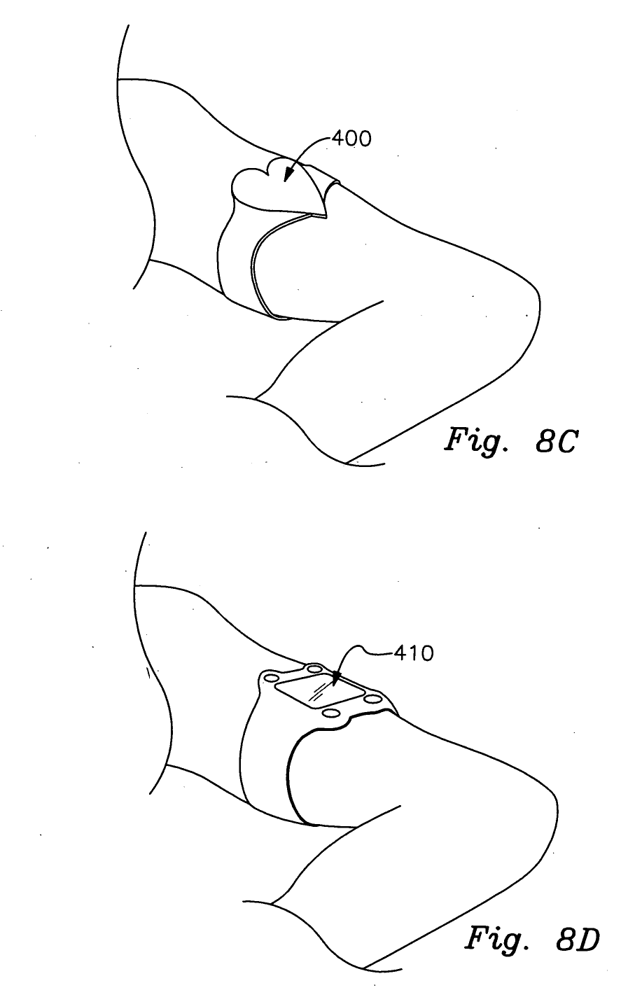

A monitoring system 300 may be used such as shown in FIGS. 8A-8D for example. In FIG. 8A, the patch monitor 300 may be adhered on the chest of the wearer, and a wrist worn device 350 may be used to receive heart rate information from the monitoring device 300. The monitoring device 300 may also be worn at other locations such as on the arm as shown in FIG. 8B. As an alternative to the adhered monitoring device system 300, the rate monitoring system to transmit information to a wrist worn device or the like, may include a strap or positioning on the arm as shown in the device 400 as shown in FIG. 8C, or the entire monitoring system, processing system and alarm indicators, such as similar to the system shown in FIG. 1, may be worn at another location, such as a device 410 worn on the arm as shown in FIG. 8D.

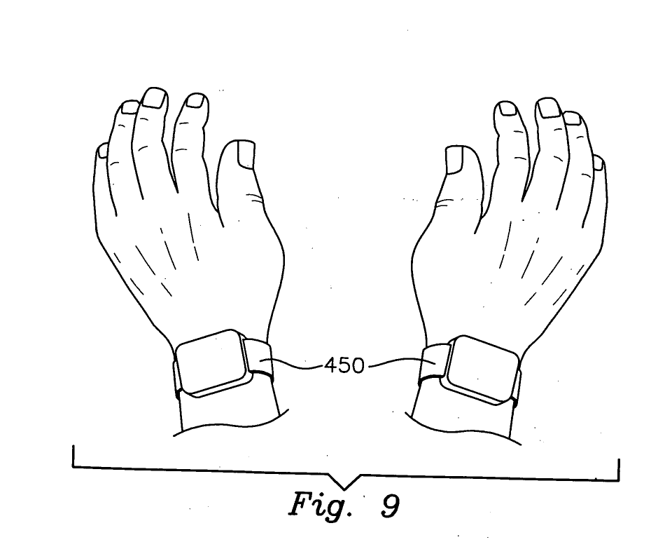

Another embodiment is shown in FIG. 9, two or more monitoring devices 400 may be worn to detect one or more physiological functions. It may be possible for example, to monitor heart rate via the two devices 450, with the polarized and non-polarized electrical heart signals detected by the alternate devices 450 to accurately monitor the heart rate.

According to a further example of the invention, the use of RFID technology or like technology may be incorporated into the devices according to the invention, for a variety of other purposes. For example, technology and systems developed by Guest Technologies, such as described in U.S. Pat. Nos. 7,147,149, 7,114,647, 7,030,765, 6,747,562, and 6,424,264 describe various functions and attributes which may be usable, with these patents hereby incorporated by reference. The Guest Technologies system may be implemented at a water park or other aquatic environment for example, to pinpoint the location of any person wearing the monitoring device. Each person is issued a monitoring device that allows them to be tracked. The monitoring devices may include locator devices that are scanned and assigned to each person, such that when they are turned on, finding a person is possible by being in proximity to readers built into interactive kiosks situated around the facility. For example, the system can recognize you and show you where the other members of your party are on a large, interactive map and display screen. The system can also help guests to navigate the facility, access weather information, access detailed resort information, and even leave messages for anyone in their family or group. Water park resorts can also do much more with this technology, including, eliminating tickets by providing the monitoring device as the admission ticket. This reduces the cost of tickets and helps keep non-paying guests out of the park. It's a bonus for guests, too, who no longer have to worry about keeping track of a ticket stub or getting their hands stamped to leave and return to the facility. The system effectively can offer an enhanced communications tool, and can also be used as a cashless payment system. The monitoring devices can be preloaded and used as a payment system. This is especially useful when all you're wearing is a bathing suit, or in other cases where carrying cash, coins, or a credit card is inconvenient. The system can be preloaded with a monetary amount, and thereafter used in the facility to get food, merchandise or to spend it any way a person wants in the facility. A variety of other applications and uses are also contemplated. In this manner, water or other parks and resorts can utilize an RFID system to enhance the usability and operation of the systems according to the invention.

Although the invention has been shown and described with reference to a various examples, it will be understood by those skilled in the art that changes or modifications in form and detail may be made without departing from the spirit and scope of the invention.

What is claimed is:

1. A drowning prevention system comprising,
a wearable article having at least one sensing system for at least one physiological function of a persons body and/or environmental information or condition around or related to the persons body, and a processing system to identify predetermined variances in the monitored at least one physiological function and/or environmental information or condition related to an emergency condition and possible drowning, and an alarm indicator for issuing an alarm if an emergency condition is detected.

2. A drowning prevention system according to claim 1, further comprising,
an alarm receiving system associated with the body of water in which the person is swimming.

3. A drowning prevention system according to claim 2, further comprising,
the alarm receiving system having an alarm indicating system for issuing a further alarm upon receipt of an alarm from a wearable article.

4. A drowning prevention system according to claim 1, wherein,
the at least one sensing system for at least one physiological function of a persons body and/or environmental information or condition are selected from one or more of the group consisting of pulse or heart rate, peripheral vasoconstriction, blood pressure, blood oxygen, CO2 blood level, body or environment temperature, body sounds or combinations thereof.

5. A drowning prevention system according to claim 1, wherein,
the at least one alarm indicator is selected from one or more of the group consisting of audible alarm, visual alarm, ultrasonic alarm, wireless signal alarm or combinations thereof.

6. A drowning prevention system according to claim 2, wherein,
the alarm receiving system being selected from one or more of the group consisting of an array of hydrophones, an array of microphones, an array of ultrasonic receivers, a visual alarm receiving system, a wireless signal receiving system or combinations thereof.

7. A drowning prevention system according to claim 2, wherein,
the alarm receiving system comprises a plurality of alarm receivers disposed in and/or around a body of water.

8. A drowning prevention system according to claim 1, further comprising,
a position detection system associated with the wearable article.

9. A drowning prevention system according to claim 1, further comprising,
a sound monitoring system associated with the wearable article.

10. A drowning prevention system according to claim 1, further comprising,
a user actuable panic switch associated with the wearable article.

11. A drowning prevention system according to claim 1, further comprising,
a user actuable reset switch associated with the wearable article.

12. A drowning prevention system according to claim 1, further comprising,
a user actuable panic switch associated with the wearable article.

13. An asphyxiation prevention system comprising,
a wearable article having at least one sensing system for at least one physiological function of a persons body and/or environmental information or condition around or related to the persons body, and a processing system to identify predetermined variances in the monitored at least one physiological function and/or environmental information or condition related to an emergency condition and possible asphyxiation, and an alarm indicator for issuing an alarm if an emergency condition is detected.

14. A heart rate monitoring system comprising,
a wearable article having at least one sensing system for sensing the electrical activity of the heart of the wearer, with the wearable article including an adhesive portion to allow it to be adhered to a users skin, and a processing system to at least monitor the users heart rate and communicate at least the heart rate information to a separate wearable article.
